(19) United States
(12) Patent Application Publication
(10) Pub. No.: US 2003/0040690 A1
(43) Pub. Date: Feb. 27, 2003

(54) Wound dressing

(76) Inventors: Wai Yuen John Chen, Wilmslow (GB); Ryan Moseley, Caerau Maesteg Mid-Glamorgan (GB); Rachel Jane Waddington, Cardiff (GB); Michael Walker, Brynford (GB)

Correspondence Address: ALLEN BLOOM, C/O DECHERT, PRINCETON PIKE CORPORATION CENTER, P.O. BOX 5218, PRINCETON, NJ, 08543-5218, US

(21) Appl. No.: 10/107,964

(22) Filed: Mar. 27, 2002

(30) Foreign Application Priority Data
Mar. 27, 2001 (GB) 0107653.8

(51) Int. Cl. A61F 13/00; A61F 13/15

(52) U.S. Cl. 602/43; 602/45; 604/368

(57) ABSTRACT

Use of a polymer which is swellable in aqueous media for the manufacture of a wound dressing comprising the polymer to reduce the concentration of superoxide radical in a wound by application of the wound dressing externally thereto. This is believed to reduce the effect of at least certain types of ROS in frustrating the healing of chronic wounds.

This invention relates to the dressing of wounds; more particularly, the present invention relates to the dressing of chronic wounds using materials not customarily regarded as pharmaceutical agents.

It is known that, in chronic wounds, a large number of endogenously produced factors are released into the wound. It is believed by some workers in this field that the abnormalities of tissue repair demonstrated in chronic wounds may, at least in part, be due to the overactivity of tissue degradation mechanisms mediated by some of these factors. In particular, there is some evidence to suggest that reactive oxygen species (ROS), such as the superoxide radical (O₂.⁻) and the hydroxyl radical (.OH), derived from polymorphonuclear leukocytes may be implicated in the pathogenesis of chronic wounds. Thus, it is believed that the presence of ROS may result in depletion of cutaneous antioxidants in the wound and that excess ROS, produced in the wound, causes extensive extracellular matrix degradation to dermal components such as collagens, proteoglycans and hyaluronan. The reactive oxygen species may also affect the metabolism of cells responsible for the synthesis of such components.

This invention seeks to reduce the effect of at least certain types of ROS in frustrating the healing of chronic wounds.

According, therefore to one aspect of this invention, there is provided use of a polymer which is swellable in aqueous media for the manufacture of a wound dressing comprising the polymer to reduce the concentration of superoxide radical in a wound by application of the wound dressing externally thereto.

This invention also provides the use of a polymer as herein defined as a wound dressing agent.

This invention further provides a method of treatment of the human body, which method comprises applying externally to a wound on the body a wound dressing which comprises a polymer which is swellable in aqueous media, the application thereby reducing the concentration of superoxide radical in the wound.

In accordance with these aspects of the invention the polymer may be a substituted or unsubstituted, homo- or co-polysaccharide. Suitably, the polymer may comprise uronic acid groups. Desirably, the substituted polysaccharide may comprise etherified or acylated hydroxyl groups, and/or may comprise esterified uronic groups. In particular, the substituted polysaccharide may comprise at least some hydroxyl groups which have been replaced by amino or acylated amino groups.

The aforementioned substituent may comprise a saturated or unsaturated, carbocyclic or heterocyclic, mono or polycyclic group. The unsaturated group may include an aromatic group. Suitably, the polycyclic group may comprise a fused polycyclic structure.

It is preferred that the weight average molecular weight of the polymer may be from 200 kDa to 200 kDa, preferably from 300 kDa to 1000 kDa. The polymer may comprise a substituted hyaluronan or a substituted cellulose. The polymer may be crosslinked. It may be formed as a film or as a fibre. Furthermore, a mixture of polymers as herein defined may be used in the manufacture of the wound dressing. When the polymer, or at least a component in a mixture of polymers, is formed as a fibre, the fibres may be disposed in the wound dressing manufactured therefrom as a non-woven mat or as a woven fibre. The materials in the wound dressing manufactured therefrom may be associated with one or more non-water swellable materials.

This invention also provides a method of treatment of the human body, which method comprises applying externally to a wound on the body a wound dressing which comprises a polymer selected from the group consisting of esterified hyaluronans, etherified celluloses and acylated celluloses, the polymer having a weight average molecular weight from about 200 kDa to about 2000 kDa, the application thereby reducing the concentration of superoxide radical in the wound.

While it is intended that the wound dressings in accordance with the present invention may be used without preservatives or pharmacologically active ingredients, it is possible to include these in minor amount. For example, an antibiotic or antimicrobial agent such as metronidazole, silver sulphadiazine, neomycin or penicillin; antiseptic agents such as povidone iodine; antiinflammatory agents such as hydrocortisone or triamcinolone acteonide; or skin protective agents such as zinc oxide may be included.

The following Figures illustrate the invention.

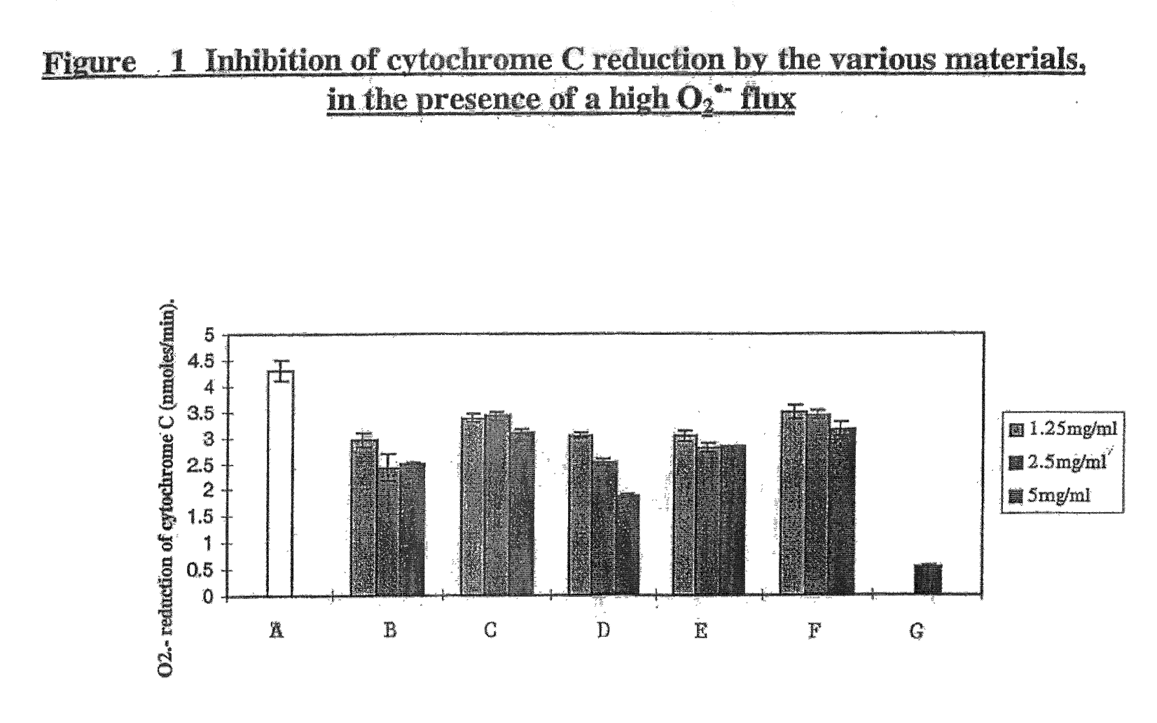

FIG. 1 shows the inhibition of cytochrome C reduction by various materials in the presence of a high O₂.⁻ flux.

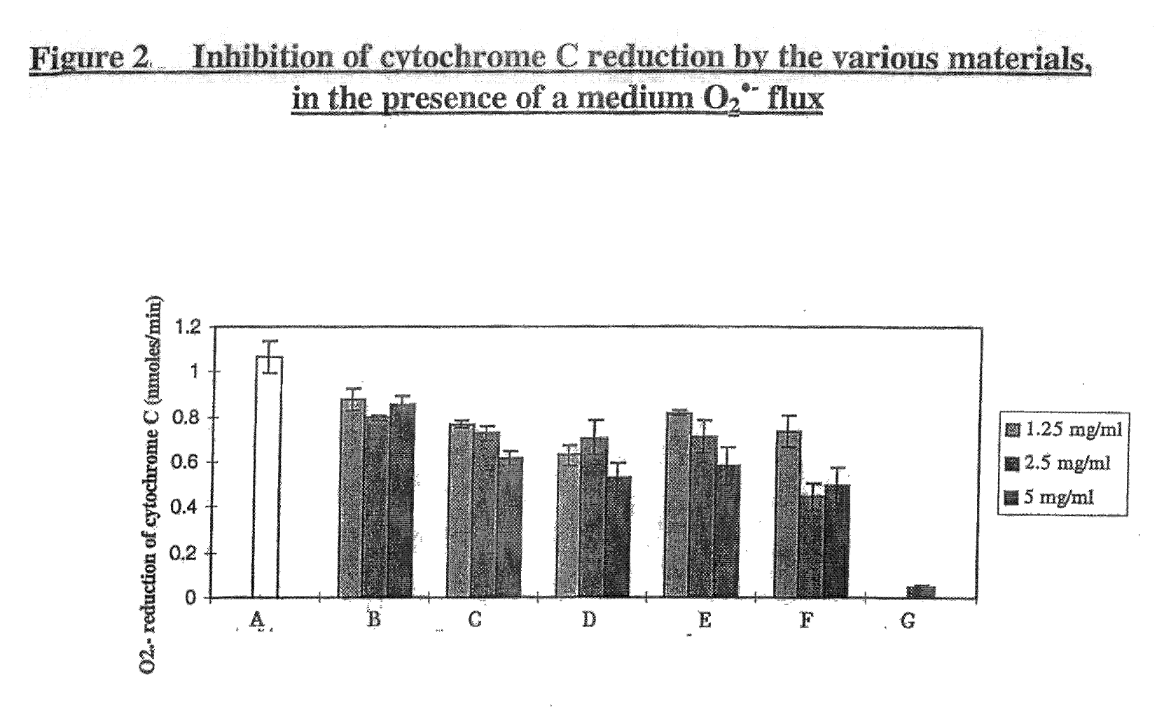

FIG. 2 shows the inhibition of cytochrome C reduction by various materials in the presence of a medium O₂.⁻ flux.

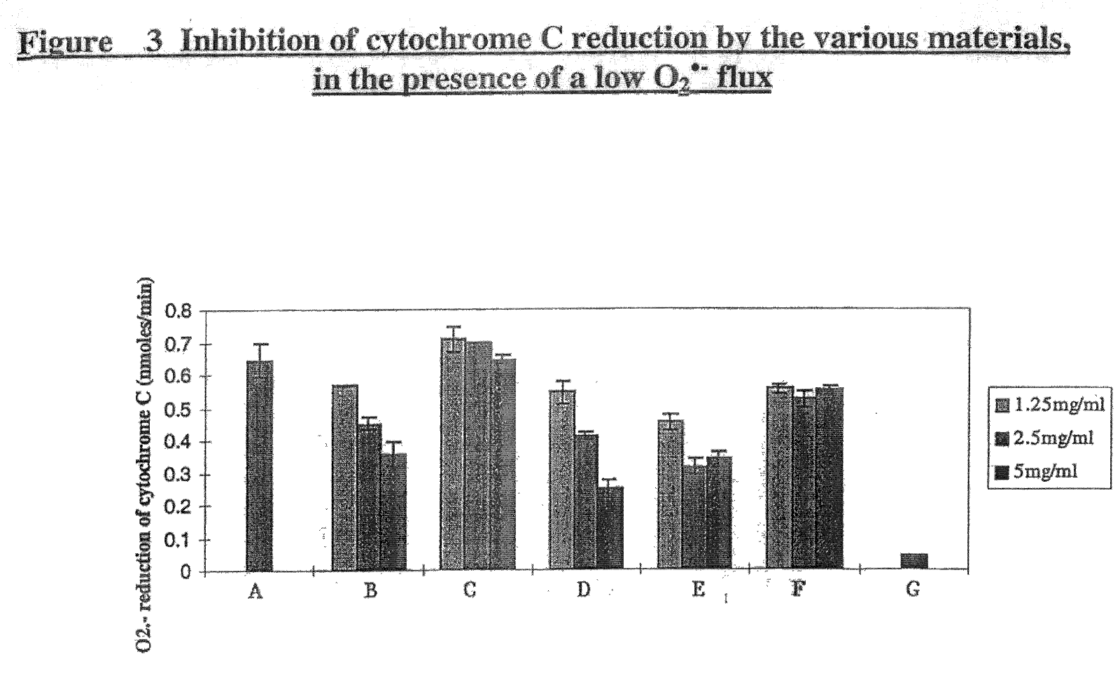

FIG. 3 shows the inhibition of cytochrome C reduction by various materials in the presence of a low O₂.^{- flux.}

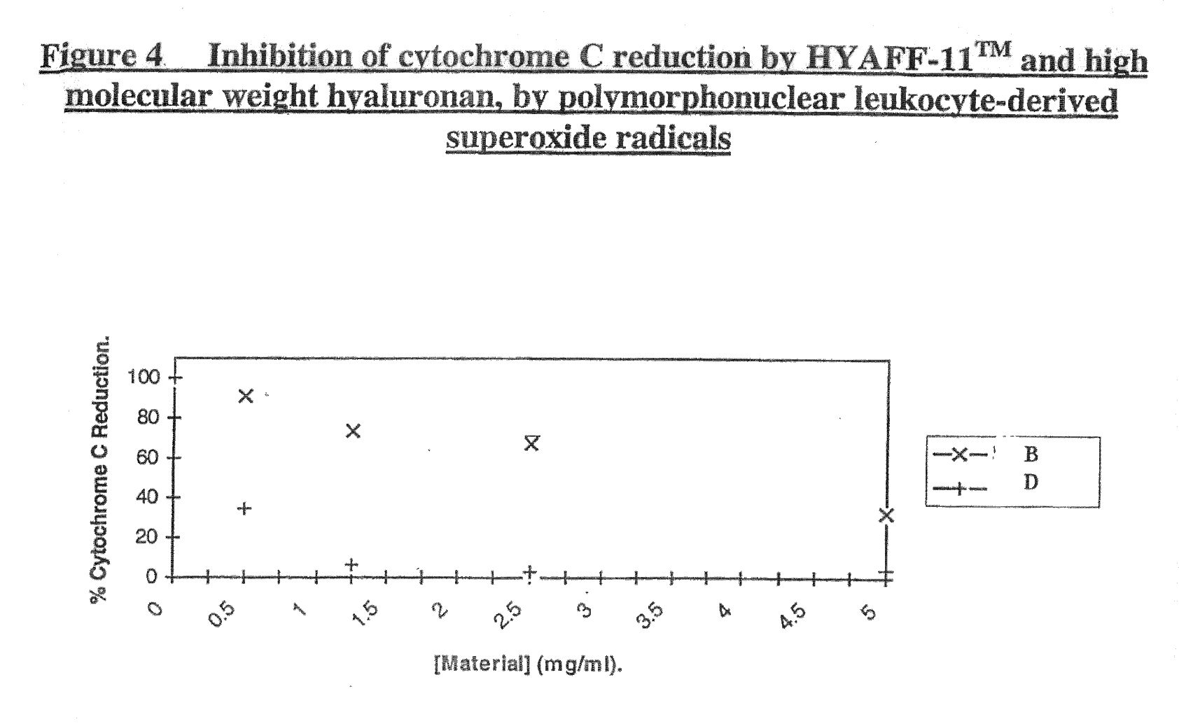

FIG. 4 shows the inhibition of cytcochrome C reduction by HYAFF-11™ and high molecular weight hyaluronan, by polymorphonuclear leukocyte-derived superoxide radicals

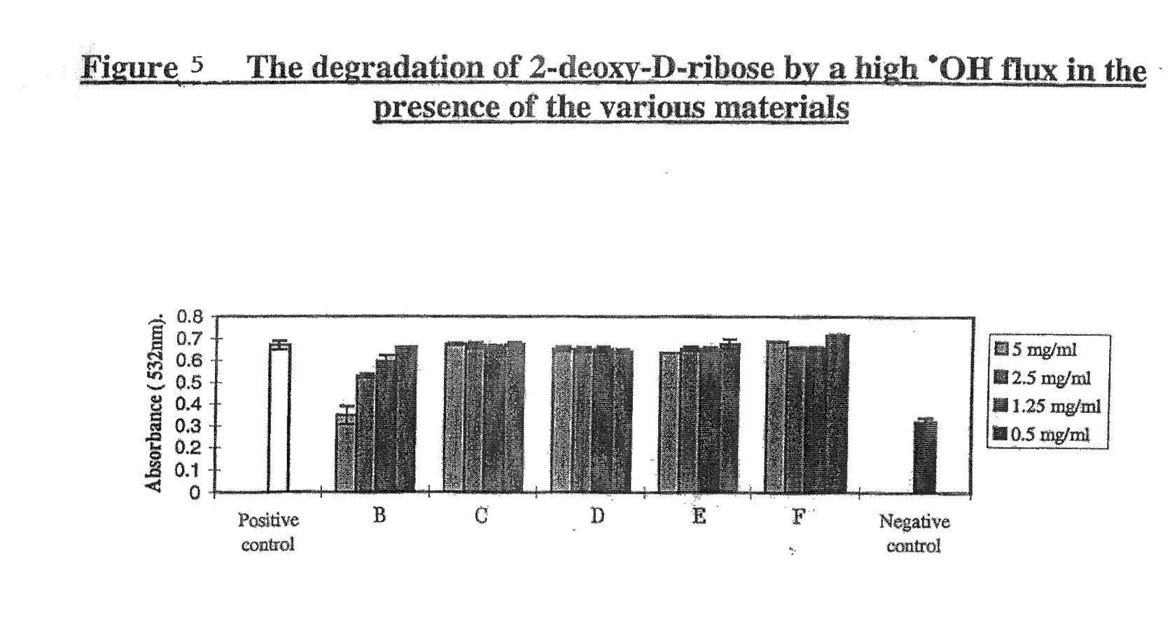

FIG. 5 shows the degradation of 2-deoxy-D-ribose by a high .OH flux in the presence of various materials.

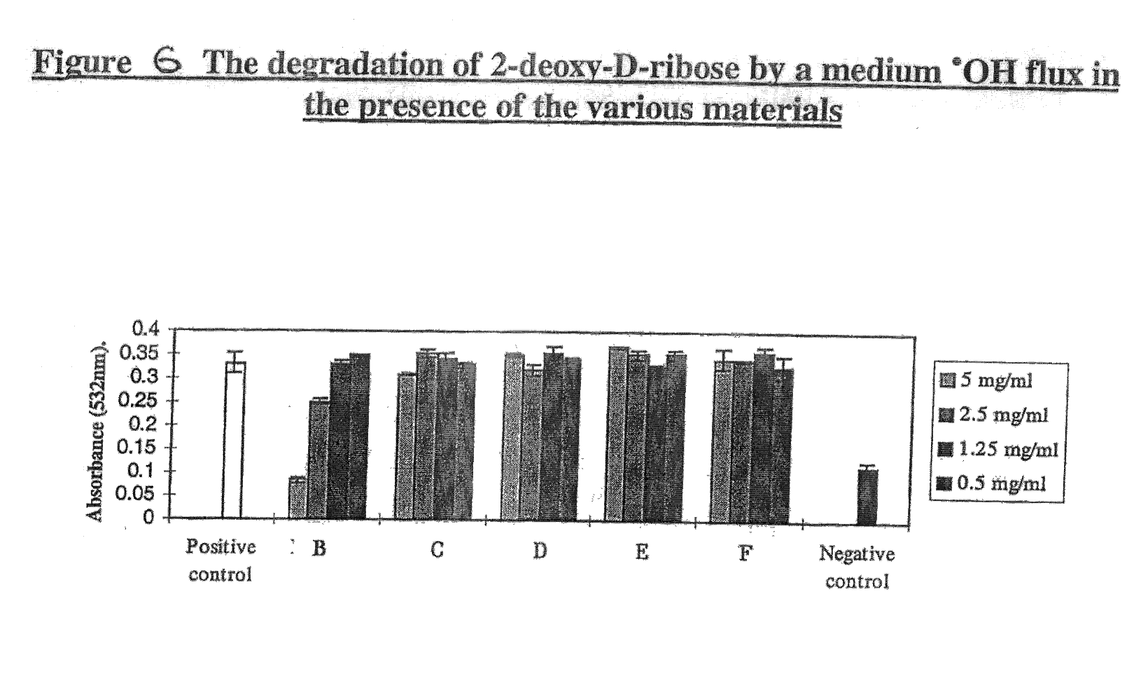

FIG. 6 shows the degradation of 2-deoxy-D-ribose by a medium .OH flux in the presence of various materials.

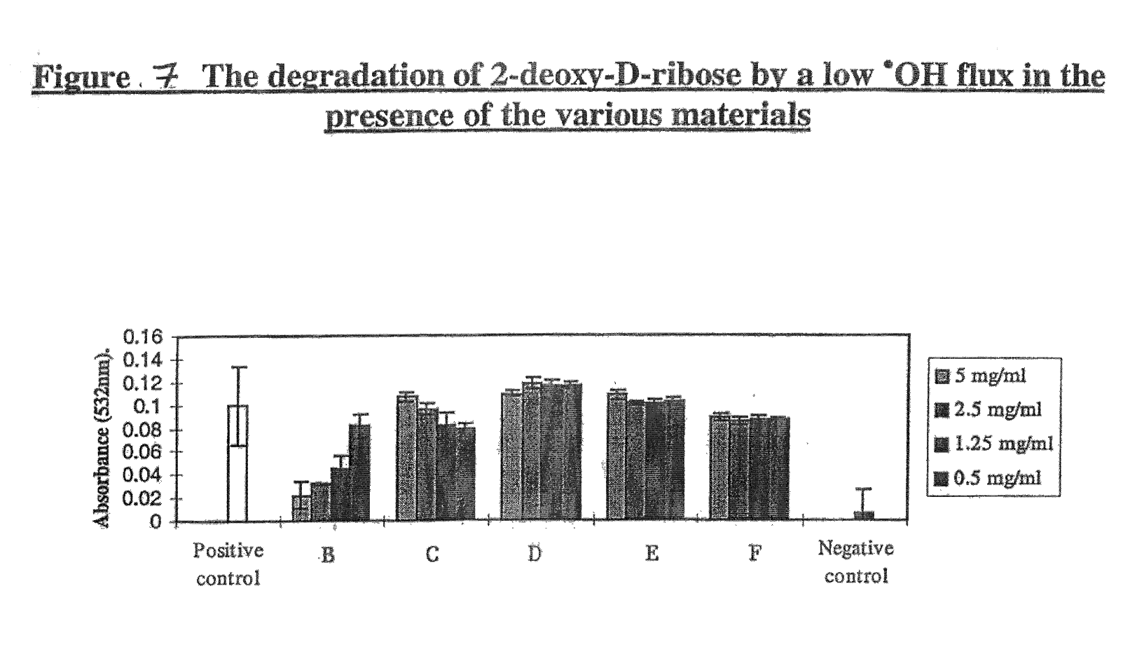

FIG. 7 shows the degradation of 2-deoxy-D-ribose by a low .OH flux in the presence of various materials.

The following Examples illustrate the invention.

### EXAMPLE 1

This Example compares the antioxidant abilities of different test materials in relation to superoxide radicals generated by cell-free systems. In the several runs of the Example, a flux of superoxide radicals was generated by oxidation of hypoxanthine by xanthine oxidase. The rate of production of the superoxide radical was measured by permitting it to reduce added cytochrome C. This reduction:

Cytochrome C(Fe⁺³)+O₂.⁻→ Cytochrome C(Fe⁺²)+O₂

produces a characteristic shift in the cytochrome C spectrum at 550 nm; and the rate of production of the superoxide radical can be determined spectrophotometrically, assuming the above stoichiometry, from the Beer-Lambert law and a molar extinction coefficient of 21,000 cm/moles/L. This is based on the assumption that one mole of radical reduces one mole of cytochrome C in accordance with the above equation. The abilities of the test materials to scavenge superoxide radicals can be determined from any decrease in the amount of reduced cytochrome C obtained in their presence.

In a total volume of 1 ml of 50 mM potassium phosphate buffer, pH 7.8, containing 10 mM EDTA, three differing fluxes of superoxide radical were generated by the oxidation of hypoxanthine (ex Sigma) by xanthine oxidase (grade III from buttermilk, ex Sigma); these were:

| | |
|---|---|
| low | 1 mM hypoxanthine and 1 mU/ml xanthine oxidase |
| medium | 2 mM hypoxanthine and 2 mU/ml xanthine oxidase |
| high | 10 mM hypoxanthine and 10 mU/ml xanthine oxidas |

Each reaction mixture also contained 10 μM cytochrome C (horse heart type III, ex Sigma).

To determine the antioxidant properties of each test material, at each of the abovementioned fluxes of superoxide radical, like reaction mixtures were next prepared but which also contained the test material, identified below, at concentrations of 1.25; 2.5 or 5 mg/ml for each test material examined.

A control reaction mixture containing 40 U/ml superoxide dismutase (SOD) (bovine erythrocytes, ex Sigma) was also established at each of the above-mentioned fluxes of superoxide radical.

Each experiment was performed in triplicate.

Upon initiation of the superoxide radical fluxes, the reduction of cytochrome C was monitored spectrophotometrically at room temperature, using a LKB Biochem Ultrospec plus 4054 UV/Visible spectrophotometer at 550 nm (ex Amersham Pharmacia Biotech) against blank reaction mixtures containing all reagents except xanthine oxidase. The absorbance values were read at 20 sec intervals for 200 secs and the relative rates of production of the superoxide radical were calculated.

The results are shown in FIGS. 1; 2 and 3 in which the test materials are identified as follows:

A: control containing hypoxanthine and xanthine oxidase only;

B: high molecular weight hyaluronan (Mw˜3 to 6000 kDa, human umbilical cord, ex Sigma);

C: low molecular weight hyaluronan (Mw˜300 kDa, bovine vitreous humor, ex Sigma);

D: HYAFF-11 (65% by weight benzylated ester of hyaluronan, Mw˜300 kDa);

E: carboxymethyl cellulose (Mw˜1000 kDa, AQUACEL ex ConvaTec Ltd);

F: a 1% solution of high molecules weight hyaluronic acid (Mw˜IOOOkDa) placed onto E (Mw˜1000 kDa, ex ConvaTec Ltd), thereby creating a thin film of approximately 500 μm;

G: 40 μl of SOD, a positive control.

HYAFF and AQUACEL are registered trade marks.

The result for each test material is in the form of bars showing the reduction in n moles/min at increasing concentration of test material. Thus for B in FIG. 1 the left hand bar shows the reduction at 1.25 mg/ml of test material, the central bar shows the reduction at 2.5 mg/ml and the right hand bar shows the reduction at 5 mg/ml.

It will be apparent that all of the test materials showed antioxidant behaviour toward the superoxide radical. This behaviour appears both molecular weight and dose dependent. Upon examination of the general trends of antioxidant activity of the test materials, D appeared to be the most effective towards the superoxide radical followed by E, B and F. All of these test materials exhibited greater antioxidant properties towards the superoxide radical than C.

The reduction of cytochrome C by the superoxide species was confirmed by the ability of SOD, at all of the fluxes of superoxide radical studied, extensively to reduce the rates of cytochrome C reduction b the superoxide radical; SOD is a known scavenger for the superoxide radical.

### EXAMPLE 2

This Example compares the antioxidant abilities of certain of the test materials used in the last Example in relation to superoxide radicals generated by isolated polymorphonuclear leukocytes.

Whole blood (25 ml) was removed from healthy human volunteers (age range 20 to 30 years) into vacutainers containing EDTA (ex Becton Dickinson, Meylan Cedex, France) as anticoagulant. Blood aliquots (2.5 ml) were then layered onto a Ficoll-Hypaque density gradient which consisted of i) a dense Ficoll-Hypaque layer (2.5 ml) comprising 9.5% by weight Ficoll 400 (ex Amersham Pharmacia Biotech) and 17% by weight Hypaque solution (sodium diatrizoate ex Sanofi Winthrop) and ii) a light Ficoll-Hypaque layer (2.5 ml) comprising 8.17% by weight Ficoll 400 and 10% by weight Hypaque solution. Tubes were next centrifuged at 2500 rpm for 45 minutes at room temperature and this treatment resulted in the formation of four layers. The two upper layers, consisting of plasma and lymphocytes/monocytes, respectively, were discarded. Each third layer (which contained the polymorphonuclear leukocytes) was removed and the several such third layers were pooled into a separate tube. The fourth layer (which comprised erythrocytes) was also discarded.

Equal volumes of phosphate buffered saline were added to the pooled polymorphonuclear leukocyte layers which were then centrifuged at 2000 rpm for 5 minutes at room temperature. The centrifuging was repeated and the polymorphonuclear leukocyte pellet was resuspended in RPMI-1640 medium (phenol red free ex Gibco), supplemented with L-glutamine (2 mM), prior to cell counting. After cell counting (1×10⁶ cells/ml), reaction mixtures were established by also including in a total volume of 1 ml (1 mg/ml) cytochrome C (horse heart type 111, ex Sigma).

To determine the antioxidant properties of each test material like reaction mixtures (1 ml) were next prepared but which also included either material D or material B at concentrations of 0.5; 1.25; 2.5 or 5 mg/ml. A control reaction mixture containing 40U/ml SOD (bovine erythrocytes ex Sigma) was also established for each material.

1 μg/ml N-formylmethionyteucyphenylatamine (FMLP) (ex Sigma) was then added to each reaction mixture to initiate the respirator burst. The reaction mixtures were next incubated at 37° C. for 10 minutes and the reaction terminated by placing reaction mixtures on ice and centrifuging at 12500 rpm for 25 minutes at 4° C. The reduction of cytochrome C by polymorphonuclear leukocyte-derived superoxide radical was measured by analogy to the procedure in Example 1, blank reaction mixtures containing all reagents except FMLP.

The results are shown in FIG. 4. It will be apparent that both materials tested exhibited significant, dose dependent antioxidant activity towards the superoxide radical, with material D being the more effective.

### COMPARATIVE EXAMPLE

This Example compares the antioxidant abilities of the same materials as were tested in the Examples of the invention in relation to hydroxyl radicals generated by cell-free systems. In the several runs of the comparative Example, a flux of hydroxyl radicals was generated by the Fenton reaction:

Fe⁺²+H₂O₂→.OH+OH⁻+Fe⁺³

The rate of production of the hydroxyl radical was measured by permitting it to degrade 2-deoxy-D-ribose to malondialdehyde and then reacting the malondialdehyde with thio-barbituric acid, under acid conditions, to form a chromogen with an absorption maximum at 532 nm. This permits spectrophotometric determination essentially as before.

In a total volume of 1 ml of 0.1 M potassium phosphate buffer, pH 7.4, containing 0.15M sodium chloride, three differing fluxes of hydroxyl radical were generated by the reaction of H₂O₂, and Fe⁺²; these were:

| | |
|---|---|
| low | 45 μM H₂O₂ and 5 μM Fe₂SO₄ |
| medium | 90 μm H₂O₂ and 10 μM Fe₂SO₄ |
| high | 180 μM H₂O₂ and 20 μM Fe₂SO₄ |

To determine the antioxidant properties of each test material, at each of the above-mentioned fluxes of hydroxyl radical, like reaction mixtures were next prepared but which also contained the test material, identified above, at concentrations of 0.5; 1.25; 2.5 or 5 mg/ml for each test material examined.

A control reaction mixture containing 10 mM thiourea (ex Sigma was also established at each of the above-mentioned fluxes of hydroxyl radical: thiourea is a known scavenger of hydroxyl radical.

The reaction mixtures were then incubated at 37° C. for 1 hour and centrifuged at 12500 rpm for 10 minutes at room temperature. 250 μL alignots of each reaction mixture of each reaction mixture were next added to 2.8% trichloracetic acid (250 μl) and 10% of thiobarbituric acid in 50 mM sodium hydroxide (250 μl). The assay mixtures were then heated at 100° C. for 15 minutes and cooled.

Each experiment was performed in triplicate. The spectrophotometric determination was effect essentially as before against blank reaction mixtures containing all reagents thiobarbituric acid.

The results are shown in FIGS. 5; 6 and 7. The result for each test material is in the form of bars showing the degradation of 2-deoxy-D-ribose by hydroxyl radical at varying concentrations of the test material. Thus for B in FIG. 5 the far left hand bar shows the degradation at 5 mg/ml of test material, the concentration of test material decreasing so that the far right hand bar shows the degradation at 0.5 mg/ml of test material.

It will be apparent from these results that the only material to possess significant, dose dependent antioxidant properties towards the hydroxyl radical is B.

It is clear from the results of these Examples that the effect shown for all materials (other than B) is highly specific to the superoxide radical.

1. Use of a polymer which is swellable in aqueous media for the manufacture of a wound dressing comprising the polymer to reduce the concentration of superoxide radical in a wound by application of the wound dressing externally thereto.

2. Use according to claim 1 wherein the polymer is a substituted or unsubstituted, homo- or co-polysaccharide.

3. Use according to claim 1 wherein the polymer comprises uronic acid grou

4. Use according to any preceding claim wherein the substituted polysaccha comprises etherified or acylated hydroxyl groups.

5. Use according to any preceding claim wherein the substituted polysaccha comprises esterified uronic acid groups.

6. Use according to any preceding claim wherein the substituted polysaccharide comprises at least some hydroxyl groups which have been replaced by amino or acylated amino groups.

7. Use according to any preceding claim wherein the substituent comprises saturated or unsaturated, carbocyclic or heterocyclic, mono or polycyclic group.

8. Use according to claim 7 wherein the unsaturated group includes an aro

9. Use according to claim 7 wherein the polycyclic group comprises a fused

10. Use according to claim 1 wherein the weight average molecular weight of

11. Use according to claim 1 wherein the polymer comprises a substituted h

12. Use according to claim 1 wherein the polymer comprises a substituted c

13. Use according to claim 1 wherein the polymer is crosslinked.

14. Use according to claim 1 wherein the polymer is formed as a film.

15. Use according to any preceding claim wherein the polymer is formed as

16. Use according to claim 1 wherein a mixture of polymers as defined in an

17. Use according to claim 15 wherein the fibres are disposed in the wound dressing manufactured therefrom as a non-woven mat or as a woven fibre.

18. Use according to claim 1 wherein the materials in the wound dressing manufactured therefrom are associated with one or more non-water swellable materials.

19. Use of a polymer according to claim 1 as a wound dressing agent.

20. A method of treatment of the human body, which method comprises applying externally to a wound on the body a wound dressing which comprises a polymer which is swellable in aqueous media, the application thereby reducing the concentration of superoxide radical in the wound.

21. A method of treatment of the human body, which method comprises applying externally to a wound on the body a wound dressing which comprises a polymer selected from the group consisting of esterified hyaluronans, etherified celluloses and acylated celluloses, the polymer having a weight average molecular weight from about 200 kDa to about 2000 kDa, the application thereby reducing the concentration of superoxide radical in the wound.
